(19) United States
(12) Patent Application Publication
(10) Pub. No.: US 2017/0246241 A1
(43) Pub. Date: Aug. 31, 2017

(54) Methods for the Selective Treatment of Tumors by Calcium-Mediated Induction of Apoptosis

(71) Applicant: Charles E. Zeilig, Aurora, CO (US)

(72) Inventor: Charles E. Zeilig, Aurora, CO (US)

(21) Appl. No.: 15/595,805

(22) Filed: May 15, 2017

Related U.S. Application Data
(63) Continuation-in-part of application No. 14/073,850, filed on Nov. 6, 2013.
Application No. 14/073,850 is a continuation-in-part of application No. 12/911,723, filed on Oct. 25, 2010, now abandoned.
Application No. 12/911,723 is a continuation-in-part of application No. 10/558,079, filed on Nov. 22, 2005, now abandoned.
(60) Provisional application No. 60/475,063, filed on May 30, 2003.

(51) Int. Cl. A61K 38/13; A61K 31/164; A61K 31/215; A61K 31/19; A61K 33/06; A61K 31/59; A61K 31/6615; A61K 31/18; A61K 31/426

(52) U.S. Cl. CPC A61K 38/13; A61K 31/18; A61K 31/164; A61K 31/215; A61K 31/426; A61K 33/06; A61K 31/59; A61K 31/6615; A61K 31/19

(57) ABSTRACT

Tumor cells exhibit consistent abnormalities in calcium regulation. The present disclosure teaches methods by which such differences are exploited to induce Apoptosis selectively in tumor/cancer cells while sparing normal cells. These methods are based upon employing drugs that, acting in synergistic combinations, trigger selective killing of malignant cells. Since the invention is based upon fundamental cell cycle requirements, to the extent that calcium handling abnormalities are a general characteristic of the malignant state, the methods presented here are widely applicable regardless of tissue of origin and degree of cellular de-differentiation.

### RELATED APPLICATIONS

This application is a continuation in part of U.S. patent application Ser. No. 14/073,850, filed Nov. 6, 2013, entitled “Methods for the Selective Treatment of Tumors by Calcium-Mediated Induction of Apoptosis”, which application is a continuation in part of U.S. patent application Ser. No. 12/911,723, filed Oct. 25, 2010, entitled “Methods for the Selective Treatment of Tumors by Calcium-Mediated Induction of Apoptosis”, which is a continuation in part of U.S. patent application Ser. No. 10/588,079, filed Nov. 22, 2005, entitled “Methods For the Selective Treatment of Tumors by Calcium-Mediated Induction of Apoptosis,” which application claims priority to and is a 35 U.S.C. §371 national phase application of PCT/US2004/017370 (WO2004/108083), filed on Jun. 1, 2004 entitled “Methods For The Selective Treatment Of Tumors By Calcium-Mediated Induction Of Apoptosis” which claims priority to U.S. provisional application Ser. No. 60/475,063 entitled “Methods For the Selective Treatment of Tumors by Calcium-Mediated Induction of Apoptosis,” filed May 30, 2003; the entire disclosures of which are hereby incorporated by reference. Any disclaimers that may have occurred during the prosecution of the above-referenced applications are hereby expressly rescinded, and reconsideration of all relevant art is respectfully requested.

### TECHNICAL FIELD

This present disclosure is in the field of medical therapeutics, more particularly in the field of clinical treatment of malignancy and cancer therapy. The methods allow a broad range of human tumors or cancer types to be treated by selectively inducing apoptosis. Apoptosis is induced in tumors by disrupting intracellular calcium distribution in a manner that leaves normal growing or non-growing cells unharmed.

### BACKGROUND

Warburg described a metabolic “defect” in energy utilization exhibited by most cancer cells. This “defect” is now known to result from a change in mitochondrial function. Many different mutations in initial growth factor dependent pathways function to produce a state in which cells are made capable of continuously passing the Pardee Restriction Point (RP) or point of no return towards the end of the G1 phase of the cell cycle. It is demonstrated that traverse through G1 prior to this point is dependent on the continuous availability of EC (extracellular) Ca²⁺. Any growth factor requirement for passing the RP is bypassed completely by Ca²⁺-specific ionophores as long as there is a ready supply of EC Ca²⁺. Carcinogenic Phorbol analogs, which act to stimulate certain forms of Ca²⁺-dependent Protein Kinase C (PKC), can replace the growth factor requirement for crossing the RP, as long as there is sufficient EC Ca²⁺ present in the growth medium. The present disclosure teaches these steps can be short-circuited and effectively bypassed by providing a ready supply of EC Ca²⁺ consistent with the known requirement for IC (intracellular) but not EC Ca²⁺ upon passing the RP. Effectively, malignant transformation mimics the effect of Ca²⁺ ionophores and Phorbol compounds and suggests the initiating event in cancer is any mutation which produces an increased new steady state of continuous Ca²⁺ influx. In order for such cells to escape Ca²⁺-induced apoptosis, several adaptations in IC Ca²⁺-handling must occur if such a potentially cancerous cell is to survive to a detectable disease state. This does not exclude the influence of known mutations in tumor suppressor or tumor promoter genes either prior to or selected for once the initiating stimulus for malignancy occurs in exacerbating the malignant state, but these mutations must be secondary to satisfying the Ca²⁺ requirement for passing the RP.

The present disclosure teaches the use of calcium manipulation for the treatment of cancer.

### SUMMARY OF THE EMBODIMENTS

The disclosure teaches a method for treating a cancer in a patient comprising administering to said patient effective amounts of two or more drugs at concentrations which interact synergistically, that stimulate an increase in the Ca²⁺ burden of smooth endoplasmic reticulum (SER) and mitochondria. The term cancer can mean a tumor in a patient. In one embodiment, the drug concentrations are submaximal. In one embodiment, at least one of said drugs stimulates Smooth-Endoplasmic-Reticulum Ca²⁺-ATPase (SERCA) and wherein at least one of said drugs is an antagonist of SER Ca²⁺ gates.

The disclosure teaches a method for treating a tumor in a patient comprising administering to said patient effective amounts of two or more drugs at concentrations which interact synergistically, that stimulate an increase in the Ca²⁺ burden of smooth endoplasmic reticulum and mitochondria.

In one embodiment at least one of said drugs stimulates SERCA and wherein at least one of said drugs is an antagonist of SER Ca²⁺ gates.

In one embodiment at least one of said drugs is selected from the group consisting of inhibitors of SER Inositol Triphosphate (IP₃)-sensitive Ca²⁺ gates and SERCA agonists, and one of said drugs are selected from the group of drugs which are stimulators of particulate Guanylate Cyclase (pGC). In one embodiment at least one of said drugs is selected from the group consisting of inhibitors of SER IP₃-sensitive Ca²⁺ gates and agonists of SERCA and wherein at least one of said drugs is an effective elevator of cyclic Guanosine Monophosphate (cGMP) levels including activators of pGCs and inhibitors of cGMP phosphodiesterases (cGMP-PDEs).

In one embodiment at least one of said drugs is a Calmodulin (CAM) antagonist, including antagonists of the CAM targets Calcineurin/protein phosphatase 2B (PP2B) (e.g. members of the class but not limited to Cyclosporine A or the cell permeable calcineurin auto inhibitory domain poly-arginine-based polypeptide; PP2B-AIP; see Tables 1, 2, and 3 in this and all subsequent drug or chemical abbreviations for exact chemical descriptions; Cyclosporin A and PP2B-AIP, Table 1) and CAM-dependent protein kinase II (CAM-PKII), for example, members of the class but not limited to KN-62 (Table 1) and wherein at least one of said drugs is a PKC agonist (e.g. members of the class but not limited to ceramide C6; Table 1).

In one embodiment at least one of said drugs is a PKC agonist and wherein at least one of said drugs is an inhibitor of cGMP-PDEs.

In one embodiment, at least one of said drugs is a PKC agonist and wherein two additional drugs of the classes CAM-PKII antagonists and PP2B antagonists are combined, each at submaximal effective drug concentrations.

In one embodiment at least one of said drugs is a CAM-PKII antagonist and wherein at least one of said drugs is a PP2B antagonist. In one embodiment at least one of the drugs is a submaximal concentration. In one embodiment, all of the drugs are at submaximal concentration.

In one embodiment at least one of said drugs is a DNA damaging agent. In one embodiment at least one of said drugs is an anti-mitotic drug.

The disclosure teaches a method of treating a tumor in a patient comprising administering to said patient effective amounts of two or more drugs that stimulate mitochondrial Ca²⁺ loading. In one embodiment further comprising administering to said patient an effective amount of a DNA damaging agent. In one embodiment further comprising administering to said patient an effective amount of an anti-mitotic drug.

The disclosure teaches a method for treating a cancer in a patient comprising administering to said patient effective amounts of two or more drugs at concentrations which interact synergistically, that stimulate an increase in the Ca²⁺ burden of smooth endoplasmic reticulum and mitochondria, wherein the drugs comprise W-7 (Table 1) and C₆C (Table 1). In one embodiment wherein the drugs comprise PMA (Table 1) and W-7. In one embodiment the drugs comprise Ski (Table 2) and W-7. In one embodiment the drugs comprise a PP2B Antagonist and C₆C. In one embodiment the drugs comprise the PP2B Antagonist PP2B_AIP and C₆C. In one embodiment the drugs comprise Cyclosporin and C₆C. In one embodiment wherein the drugs comprise an Akt/Protein Kinase B Antagonist (e.g. Triciribine, Table 2) and C₆C. In one embodiment wherein the drugs comprise calcium, vitamin D (Table 3) and IP₆ (Table 3).

The disclosure teaches any of the methods listed above further comprising the drug Sodium di-Chloro-Acetate (DCA, Table 3).

The disclosure teaches a combination of at least two drugs for a synergistic effect. The disclosure further teaches a combination of at least three drugs for synergistic effect.

### BRIEF DESCRIPTION OF THE DRAWINGS

FIG. 1. Regulatory enzymatic tetrad controlling calcium targets and calcium distribution required for transitions between two sequential cell cycle phases.

FIG. 2. Time Course of Effect of the Calmodulin Antagonist W-7 on Induction of Apoptosis in malignant human melanocyte-derived MEL-STR Cells.

FIG. 3. Dose Response Comparison of the Effect of W-7 on Induction of Apoptosis in Malignant MEL-STR and nonmalignant human melanocyte-derived MEL-STVP Cells.

FIG. 4. Dose Response Comparison of the Effect of the dual PP2A (protein phosphatase 2A) and PKC Agonist C₆C on Induction of Apoptosis in Malignant MEL-STR and Non-Malignant MEL-STVP Cells.

FIG. 5. Selective Potentiation of Apoptosis between the Cell Permeable Auto-Inhibitory Peptide (AIP) PP2B Antagonist and the PKC/PP2A Agonist C₆C in Malignant MEL-STR but Not Non-Malignant MEL-STVP Cells.

FIG. 6. Potentiation of Apoptosis by the PP2B Calmodulin Antagonist Cyclosporin A and by the PKC/PP2A Agonist C₆C in Malignant MEL-STR Cells.

FIG. 7. Potentiation between the PKC/PP2A Agonist C₆C and the PP2B Calmodulin Antagonist W-7 on Induction of Apoptosis in Malignant MEL-STR Cells.

FIG. 8. Potentiation between a Sphingosine Kinase Antagonist, SKi (4-[[4-(4-Chlorophenyl)-1,3-thiazol-2-yl]amino]phenol), and W-7 on Induction of Apoptosis in Malignant MEL-STR Cells.

FIG. 9. Potentiation between the PKC Agonist PMA and the Calmodulin Antagonist W-7 on Induction of Apoptosis, Growth Inhibition, and Microscopic or FACS Morphology in Malignant MEL-STR Cells.

FIG. 10. Potentiation of Apoptosis and Inhibition of Growth Rate using an Akt/Protein Kinase B Antagonist (Triciribine) in Combination with the PKC/PP2A Agonist C₆C in Malignant MEL-STR Cells.

FIG. 11. Prophetic Example in a Patient Diagnosed with Prostate Cancer and Subjected to a Treatment Regimen Designed to Produce Endoplasmic Reticulum Calcium Overload Using an Over-The-Counter 3 Component Mixture of Agents.

FIG. 12. Prophetic Example in a Patient Diagnosed with Inoperable Metastasized Pancreatic Cancer with a 3 Month Survival Estimate and Subjected to a Treatment Regimen Designed to Produce Endoplasmic Reticulum Calcium Overload Using an Over-The-Counter 3 Component Mixture of Agents.

FIG. 13 Comparison of dose response relationships of C₆C on induction of apoptosis in STR and STVP cells.

FIG. 14 Comparison of dose response relationships of W-7 on induction of apoptosis in STR and STVP cells.

FIG. 15 Potentiation of apoptosis by a Calmodulin antagonist and a PKC agonist in STR cells.

FIG. 16 Potentiation of apoptosis, growth inhibition, and morphological transformation by the Calmodulin antagonist W-7 and the PKC agonist PMA in STR cells.

FIG. 17 Selective potentiation of a PP2B antagonist by a PKC agonist in transformed but not normal cells.

FIG. 18 is FIG. 2 from “N-(6-Aminohexyl)-5-chloro-1-naphthalenesulfonamide, a calmodulin antagonist, inhibits cell proliferation” (Proc. Nat. Acad. Sci. USA, Vol. 78, No. 7, pp. 4354-4357, July 1981 Cell Biology) for dose response curve from which EC50 can be read.

FIG. 19 is FIG. 3 from “A link of Ca2+ to cAMP oscillations in Dictyostelium: the calmodulin antagonist W-7 potentiates cAMP relay and transiently inhibits the acidic Ca2+-store” (BMC Developmental Biology; 4(2004) pg 7) from which EC50 can be read.

FIG. 20 is FIG. 1d from “Protein kinase C-mediated Ca2 [symbol] entry in HEK 293 cells transiently expressing human TRPV4” (British Journal of Pharmacology (2003) Volume 140, pp. 413-421) for dose response from which EC50 can be read.

FIG. 21 is FIG. 2 from “Phorbol esters, phospholipase. C, and growth factors rapidly stimulate the phosphorylation of a Mᵣ 80,000 protein in intact quiescent 3T3 cells” (Proc. Natl. Acad. Sci. USA, Vol. 80, pp. 7244-7248, December 1983, Cell Biology) for Phorbol dose response.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

### DETAILED DESCRIPTION

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Submaximal concentration is defined as a concentration of a drug that is at least 50% lower than the concentration given for the drugs maximal effect when given alone. The concentration may be 10-fold lower than its maximal effect when given alone.

Cancer cells are cells that continuously divide, forming solid tumors or with abnormal cells that not in solid tumor form. Healthy cells stop dividing when there is no longer a need for more daughter cells, but cancer cells continue to produce copies.

Drugs that are SERCA stimulators/agonists include but are not limited to: Ceramide, C2-Ceramide, C6-Ceramide, HK654, PMA, and functional equivalents thereof (see Table 1, Protein Kinase C Agonists).

Drugs that are inhibitors/antagonists of SER IP₃-sensitive Ca²⁺ gates include but are not limited to: IP6, IP5, and functional equivalents thereof (see Table 3, Endoplasmic Reticulum Ca²⁺ Overload—IP₃—Receptor Antagonists).

Drugs that are agonists (activators/stimulators) of particulate guanylate cyclases include but are not limited to: Ceramide, C2-Ceramide, C6-Ceramide, HK654, PMA, and functional equivalents thereof (see Table 1, Protein Kinase C Agonists)

Drugs that are effective elevators of cGMP levels include but are not limited to: Ceramide, C2-Ceramide, HK654, PMA, and functional equivalents thereof (see Table 1, Protein Kinase C Agonists).

Drugs that are inhibitors of cGMP phosphodiesterases include but are not limited to: Viagra, Cialis, Levitra, Sulindac (and derivatives), and functional equivalents thereof (See Table 2, Endoplasmic Reticulum^{Ca2+Overload}-cGMP PDE Antagonists).

Drugs that are calmodulin (CAM) antagonists include but are not limited to: W-7 and functional equivalents thereof (See Table 1, Calmodulin Antagonists).

Drugs that are Protein Kinase C (PKC) agonists include but are not limited to: Ceramide, C2-Ceramide, C6-Ceramide, HK654, PMA and functional equivalents thereof (see Table 1, Protein Kinase C Agonists).

Drugs that are Protein Phosphatase 2A agonists include but are not limited to: Ceramide, C2-Ceramide, C6-Ceramide, and functional equivalents thereof (see Table 1, Protein Phosphatase 2A Agonists).

Drugs that are CAM-dependent protein kinase II antagonists include but are not limited to: CK59, KN-93, KN-62, and functional equivalents thereof (see Table 1, Calmodulin-dep. Protein Kinase—II Antagonists).

Drugs that are Calcineurin/CAM-dependent protein phosphatase 2B antagonists include but are not limited to: CN585, Cell Permeable Calcineurin Auto inhibitory Peptide, Cyclosporin A, FK-506, and functional equivalents thereof (see Table 1, Calmodulin-dep. Protein Phosphatase 2B Antagonists).

Drugs that are Warburg Metabolic Antagonists include but are not limited to: Various salts of DCA, and functional equivalents thereof (see Table 3, Warburg Metabolic Antagonists).

Drugs that are DNA damaging agents include but are not limited to: Ara-C I[Cytosine β-D-arabinofuranoside] and functional equivalents thereof.

Drugs that are anti-mitotic drugs include but are not limited to: Vinblastine. [dimethyl (2β,3β,4β,5α,12β,19α)-15-[(5S,9S)-5-ethyl-5-hydroxy-9-(methoxycarbonyl)-1,4,5,6,7,8,9,10-octahydro-2H-3,7-methanoazacycloundecino[5,4-b]indol-9-yl]-3-hydroxy-16-methoxy-1-methyl-6,7-didehydroaspidospermidine-3,4-dicarboxylate] and functional equivalents thereof.

The EC50 is the concentration of a drug that gives half-maximal response. The IC50 is the concentration of an inhibitor where the response (or binding) is reduced by half. EC stands for “Effective Concentration” and IC stands for “Inhibitory Concentration”. The EC50 can easily be determined from dose response curves.

The disclosure teaches regulation of cell cycle traverse involved a series of alternating switches consisting of elevated cGMP, Ca²⁺ uptake and sequestration within the ER, and reduced cytosolic [Ca²⁺ ]. These phases are followed by periods of elevated cAMP, release of ER^{Ca2+,increased} cytosolic [Ca²⁺], and net^{Ca2+efflux} from the cell. Some of these switches correlate with known cell cycle transitions. The correlated cell cycle phenomena include the relationships between the Cyclin Kinase and calcium regulatory systems. This system is known as Calcium Storage/Release Hypothesis of Cell Cycle Regulation (manuscript in preparation). Cytosolic [Ca²⁺ ] is measured in synchronized cells and is in agreement, quantitatively and temporally. The relationships between calcium, cyclic nucleotides, Cyclin Kinases, and checkpoint control systems, are used for the treatment of cancer.

The disclosure teaches uses for predicting new avenues for treating malignancy and it has been tested experimentally with positive results. The disclosure teaches an approach that is generalizable in many cancers, as it is based on one fundamental cell cycle aberration common to most if not every form of cancer. Cancers include but are not limited to melanoma, prostate, pancreatic, breast, lymphoma, lung, colon, etc.

The Warburg effect is a metabolic “defect” in energy utilization exhibited by most cancer cells. This so-called “defect” results from a change in mitochondrial function. This disclosure teaches that this “defect” is not really a defect at all but rather is a normal process that is shared by other very rapidly growing cell such as early embryonic cells. This disclosure teaches that malignant cells merely co-opt an existing system which somehow is consistent with or enables rapid proliferation.

Many different mutations in initial growth factor dependent pathways function to produce a state in which cells are made capable of continuously passing the so-called Pardee Restriction Point (RP) or point of no return towards the end of the G1 phase of the cell cycle. Traversal through G1 prior to this point is dependent on the continuous availability of EC Ca²⁺. Any growth factor requirement for passing the RP is bypassed completely by Ca²⁺-specific ionophores as long as there is a ready supply of EC Ca²⁺. Carcinogenic Phorbol analogs, which act to stimulate certain forms of Ca²⁺-dependent Protein Kinase C isoforms (PKC), can replace the growth factor requirement for crossing the RP, as long as there is sufficient EC Ca²⁺ present in the growth medium. This disclosure teaches that for a normal cell to become irreversibly committed to pass through the cell cycle, these steps are effectively bypassed by providing a ready supply of EC Ca²⁺ consistent with the known requirement for IC but not EC Ca²⁺ upon passing the RP. Malignant transformation mimics the effect of Ca²⁺ ionophores and Phorbol compounds and the initiating event in cancer is any mutation which produces an increased new steady state of continuous Ca²⁺ influx. In order for such cells to escape Ca²⁺-induced apoptosis, several adaptations in IC Ca²⁺-handling occur if such a potentially cancerous cell is to survive to a detectable disease state. This does not exclude the influence of known mutations in tumor suppressor or tumor promoter genes either prior to or selected for once the initiating stimulus for malignancy occurs in exacerbating the malignant state. However, all of such mutations must be secondary to satisfying the Ca²⁺ requirement for passing the RP.

This disclosure teaches the anticancer mechanism of Vitamin D is through short term elevation of Ca²⁺ availability through intestinal absorption and short increase in Ca²⁺ uptake by cancer cells. Suppression of and lower incidence of cancer occurrence requires only a slight increase in Ca²⁺ overload in malignant cells. The efficacy of Vitamin D plus Ca²⁺ supplements are potentiated by drugs designed to reduce release of Ca²⁺ from the smooth endoplasmic reticulum (SER). In one embodiment, the drug would be an antagonist of the SER IP₃ receptor.

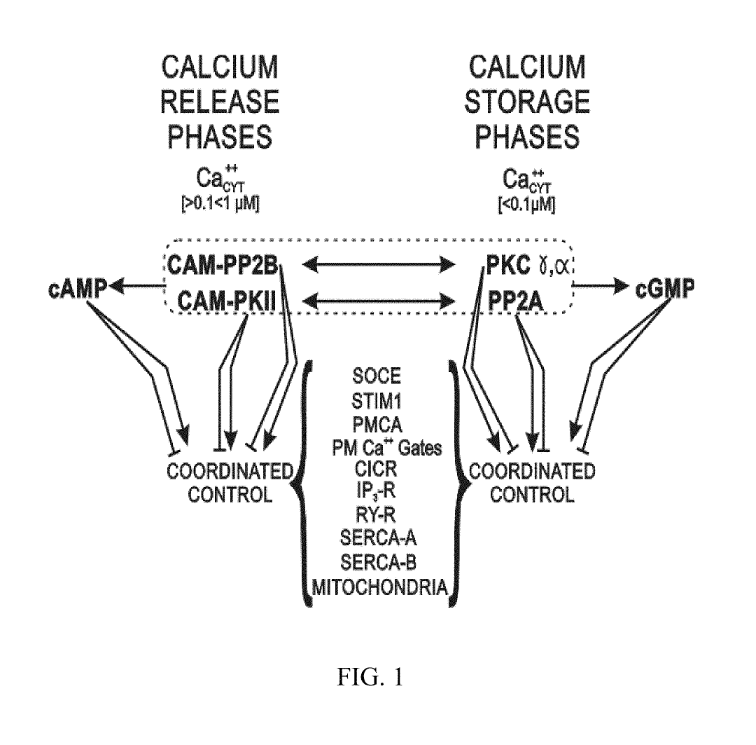

Cell cycle checkpoints occur during periods of Ca²⁺ sequestration and elevated cGMP levels. Cells can be prevented from passing out of these phases either directly or indirectly. Prolonged exposure to Ca²⁺ influx triggers apoptosis significantly more easily in cancer cells compared to normal cells. Once normal cells pass the RP, they can complete one pass through the cell cycle in the absence of external growth factors. Only the intrinsic apoptotic pathway is used to trigger apoptosis in the event of uncorrectable genetic and chromosomal errors, as governed by cell cycle checkpoints. This pathway converges on the mitochondrion and involves Ca²⁺. The mitochondrial Ca²⁺ uptake pathway normally requires facilitated transfer of Ca²⁺ directly from the SER as opposed to some cell-wide increase in Ca²⁺. This disclosure teaches the use of drugs which shift the equilibrium from SER Ca²⁺ release to SER Ca²⁺ uptake. This disclosure teaches 2 (or more) drug combinations directed against a tetrad of specific enzymes to achieve synergistic interactions and lower the possibility of unwanted side effects. Non-limiting examples of drugs are found in Table 1, 2 and 3. This tetrad and the mediators of Ca²⁺ distribution into and out of various compartments is illustrated in FIG. 1.

Three main cell cycle checkpoints coincide with Ca²⁺ storage phases. The Warburg phenomenon is related to changes in mitochondrial Ca²⁺ content. Preventing cells from passing out of the Ca²⁺ storage phases leads to mitochondrial Ca²⁺ overload and subsequent apoptosis. The Ca²⁺ regulatory enzyme tetrad is a means of not only controlling exit from Ca²⁺ storage phases but also towards a method for converting cells residing in the Ca²⁺ release phases to a state of continuous Ca²⁺ storage and ultimate apoptosis. This predicts how cancer cells can be forced to undergo apoptosis by pharmaceutical intervention of Calmodulin- and PKC/PP2A-dependent processes.

Three major “Checkpoints” have been identified which, in the face of uncorrectable errors in DNA integrity (including proper chromosomal separation at anaphase), arrest cell cycle progression and lead to apoptosis. The timing of these three Checkpoints coincides with cell cycle phases during which EC Ca²⁺ is sequestered within the SER. A fourth checkpoint is known to occur either at the end of S-Phase or before the beginning of G2 but only leads to a slowing of cell cycle traverse rather than apoptosis and does not coincide with Ca²⁺ sequestration.

The intrinsic apoptosis pathway which operates during the cell cycle depends on the transference of Ca²⁺ into the ER and ultimately into the mitochondria.

Progression of cells through the cell cycle is dependent on the ordered synthesis of specific Cyclins and activation of their partnering kinases. Likewise, cell cycle progression is also obligatorily dependent on activation of specific Ca²⁺-sensitive intracellular receptors such as Calmodulin and Ca²⁺-sensitive forms of Protein Kinase C. Errors in the operation of either of these two regulatory systems have the power to arrest cells at specific transition points in the cell cycle. These two systems function in an obligatorily inter-related manner.

Cancer cells differ from normal cells in their Ca²⁺ handling. If cells could be pharmacologically arrested in Ca²⁺-sequestering phases by interfering with Ca²⁺-dependent mechanisms necessary to transition out of these phases, it triggers apoptosis. The extra burden of sequestered Ca²⁺ in cancer cells allows for the selective induction of apoptosis in cancer cells before harming non-malignant cells. The present disclosure teaches the selective induction of apoptosis of cancer cells with reduction of toxic side-effects using novel 2 (or more)-drug combinations which are mutually synergistic.

FIG. 1. shows the Regulatory Enzymatic Tetrad Controlling Calcium Targets and Calcium Distribution in Two Different, Contiguous Cell Cycle Phases. Abbreviations used: CAM-PP2B, Calmodulin-Dependent Protein Phosphatase 2B; CAM-PKII, Calmodulin-Dependent Protein Kinase Type II; PKC, Protein Kinase C (Ca²⁺-stimulated subtypes); PP2A, Protein Phosphatase 2A; cAMP, Cyclic Adenosine Monophosphate; PKA, Cyclic AMP-Dependent Protein Kinase; cGMP, Cyclic Guanosine Monophosphate; PKG, Cyclic GMP-Dependent Protein Kinase; SOCE, Store Operated Calcium Entry; STIM 1, Stromal Interaction Molecule 1; PMCA, Plasma Membrane Calcium ATPase; PM Ca²⁺ Gates (also known as CRAC or ORAI), Ca²⁺-specific plasma membrane influx channels; CICR, Calcium-Induced Calcium Release; IP₃—R, Inositol Triphosphate Receptor; RY-R, Ryanodone Receptor; SERCA-A/B, Smooth Endoplasmic Reticulum Calcium ATPase, type A or B.

This illustration summarizes the cellular targets which regulate Ca²⁺ distribution between various compartments as cells pass from one phase or regulatory switch-point to the next during the cell cycle. Each of the Tetrad enzymes acting directly, or secondarily through cyclic nucleotide dependent protein kinases, exert highly coordinated regulation of the functional activity of targets that control movement of Ca²⁺ between cellular compartments and in and out of the cell. Of the various targets regulating Ca²⁺ movements, some are activated and some are inactivated by phosphorylation. In each case, cells proceed from one switch point to the next. These phosphorylation events are reversed by opposing phosphatases. Thus, CAM-PKII is opposed by PP2A and PKC is opposed by PP2B. Steady state levels of cytosolic Ca²⁺ vary between high and low levels for the entire length of each particular phase. These switch-points obligatorily control whether a cell will successfully transition from one phase to the next and successfully proceed through that phase. Pairs of contiguous phases are characterized by net Ca²⁺ uptake, sequestration of said Ca²⁺ into the SER compartment, and concomitant lowering of cytosolic Ca²⁺ below the CAM activation threshold ([Ca²⁺]<0.1 μM). The following phase is characterized by release of sequestered Ca²⁺ into the cytosol in coordination with activation of the PMCA efflux pump exactly balanced to elevate cytosolic [Ca²⁺ ] above the CAM activation threshold and below the PKC activation range (>0.1 μM<1.0 μM) and to gradually reduce SER-sequestered and total cellular Ca²⁺ over time.

By pharmacologically manipulating the activity of the Tetrad enzymes by appropriate stimulation or inhibition, progression through the cell cycle is arrested and all cells in the population are forced into a state of continuous Ca²⁺ accumulation. Ultimately this leads to SER and mitochondrial Ca²⁺ overload and triggering of apoptosis. Pharmacological manipulation of any pair of the Tetrad enzymes will interact synergistically to trigger an apoptotic response and thus can be used to reduce drug concentrations and toxicity clinically as well as shortening treatment duration. Apoptotic sensitivity of malignant cells to such treatments will be significantly greater than normal cells as a result of a greater burden of sequestered SER and mitochondrial Ca²⁺ in cancer cells.

In each of the treatment methods provided, there is a therapeutic window for selectively initiating an Apoptotic cascade in tumor cells without simultaneously inducing undesirable side effects in normal Ca²⁺-dependent physiological processes of normal cells. This treatment window can easily be determined by the routine experimentation of one skilled in the art. While inhibitors of plasma membrane efflux pumps may provide some clinical efficacy, employing submaximal combinations of drugs that interact synergistically to increase cellular Ca²⁺ loading provides an unexpected means to reduce undesirable side effects and to increase therapeutic indices.

The duration of treatment required to initiate an Apoptotic response in patients is relatively brief, on the order of 8 to 16 hours. In one embodiment, on the order of 3 to 6 hours. In one embodiment, 2 to 20 hours. In one embodiment, 4 to 6 hours. In one embodiment, 5 to 7 hours. Individual drugs or drug combinations are administered by standard means according to the absorptive and pharmacokinetic requirements of efficacious drug candidates. The therapeutic agents are administered orally or intravenously in amounts calculated to achieve measured blood concentrations approximating those determined to be effective from tissue culture studies. Each drug is used at the lowest dosage shown to produce mutual potentiation of apoptosis. In one embodiment, submaximal concentrations are used.

The dosage of each drug is calculated to provide clinically effective blood levels for a period of 3 to 5 hours based on animal and Phase I trials. This short duration of treatment is based upon the minimum time required to force tumor cells into irreversible commitment to apoptosis. Resorption of a patient's tumor can be followed at appropriate intervals thereafter using ultra-sensitive techniques such as PET or SPECT molecular imaging. This regimen can be repeated daily if required based upon the severity, if any, of side-effects and by the rate of tumor shrinkage. Given the thresholds of sensitivity to calcium-induced apoptosis between normal and cancerous cells, such side-effects are likely to be fairly innocuous.

Blood levels of given therapeutic agents are monitored by suitable assay methods specifically developed for this purpose in order to maximize therapeutic ratios. Depending on the severity of any side effects, this treatment regimen is repeated at regular intervals as often as necessary to maximize tumor regression. In one embodiment, drug responsiveness and treatment efficacy are monitored during the course of drug administration by assay of blood levels of apoptotic markers, namely any of several caspases released by cells undergoing Apoptosis specifically developed for this purpose. In this way, patients are spared unnecessarily prolonged drug exposure and the clinician is furnished with immediate evidence of treatment efficacy.

Tables 1, 2 and 3 list drugs for the synergistic effects as described above.

TABLE 1
PRIMARY APOPTOTIC TARGETS
TABLE 1 - PRIMARY APOPTOTIC TARGETS

| PRIMARY ENZYME TETRAD | DRUG/CHEMICAL | DRUG/CHEMICAL |
|---|---|---|
| TARGETS | COMMON NAME | CHEMICAL NAME |
| Calmodulin-dep. Protein Kinase - | CK59 | 2-(2-Hydroxyethylamino)-6-aminohexylcarbamic acid tert- |
| II Antagonists | | butyl ester-9-isopropylpurine |
| | KN-93 | 2-[N-(2-hydroxyethyl)]-N-(4-methoxybenzenesulfonyl)]amino- |
| | | N-(4-chlorocinnamyl)-N-methylbenzylamine) |
| | KN-62 | 1-[N,O-bis-(5-Isoquinolinesulfonyl)-N-methyl-L-tyrosyl]-4- |
| | | phenylpiperazine |
| Calmodulin-dep. Protein | CN585 | 6-(3,4-dichloro-phenyl)-4-(N,N-dimethylaminoethylthio)-2- |
| Phosphatase 2B Antagonists | | phenyl-pyrimidine |
| | Calcineurin Auto | 11R-CaN-AlD, Ac- |
| | inhibitory Peptide, Cell- | RRRRRRRRRRRGGGRMAPPRRDAMPSDA-NH₂ |
| | permeable | |
| | Cyclosporin A, | {R-[R*,R*-(E)]}-cyclic-(L-alanyl-D-alanyl-N-methyl-L-leucyl- |
| | Tolypocladium inflatum | N-methyl-L-leucyl-Nmethyl-L-valyl-3-hydroxy-N,4-dimethyl-L- |
| | | 2-amino-6-octenoyl-L-α-amino-butyric-N-methyl-glycinyl- |
| | | Nmethyl-L-leucyl-L-valyl-N-methyl-leucyl) |
| | FK-506, Streptomyces | (3S,4R,5S,8R,9E,12S,14S,15R,16S,18R,19R,26aS)- |
| | sp. | 5,6,8,11,12,13,14,15,16,17,18,19,24,25,26,26a |
| | | Hexadecahydro-5,19-dihydroxy-3-[(1E)-2--[(1R,3R,4R)-4- |
| | | hydroxy-3-methoxycyclohexyl]-1-methylethenyl]-14,16- |
| | | dimethoxy-4,10,12,18-tetramethyl-8-(2-propen-1-yl)-15,19- |
| | | epoxy-3H-pyrido[2,1-c][1,4]oxaazacyclotricosine- |
| | | 1,7,20,21(4H,23H) tetrone |
| Calmodulin Antagonists | W-7 | N-(6-aminohexyl)-5-chloro-1-naphthalenesulfonamide |
| | | hydrochloride |
| Protein Phosphatase 2A | Ceramide | D-erythro-Sphingosine |
| Agonists | C2-Ceramide | N-Acetyl-D-sphingosine |
| | C6-Ceramide | N-Hexanoyl-D-erythro-Sphingosine |
| Protein Kinase C Agonists | Ceramide | D-erythro-Sphingosine |
| | C2-Ceramide | N-Acetyl-D-sphingosine |
| | C6-Ceramide | N-Hexanoyl-D-erythro-Sphingosine |
| | HK654 | Diacylglycerol-lactone analog (cell permeable) |
| | PMA | Phorbol-12-Myristate-13-Acetate |

TABLE 2
SECONDARY APOPTOTIC TARGETS
TABLE 2 - SECONDARY APOPTOTIC TARGETS

| SECONDARY APOPTOTIC | DRUG/CHEMICAL | DRUG/CHEMICAL |
|---|---|---|
| TARGET | COMMON NAME | CHEMICAL NAME |
| Endoplasmic Reticulum | Ski, Ski-2 | 4-[[4-(4-Chlorophenyl)-1,3-thiazol-2-yl]amino]phenol |
| Ca2 + Overload - | | |
| Sphingosine Kinase Antagonist | | |
| Endoplasmic Reticulum | Triciribine | 6-amino-4-methyl-8-(beta.-D-ribofuranosyl)pyrrolo |
| Ca2 + Overload - | | [4,3,2-de]pyrimido[4,5-c]pyridazine |
| Akt/Protein Kinase B Antagonist | | |
| Endoplasmic Reticulum | Viagra | 1-[4-ethoxy-3-(6,7-dihydro-1-methyl- |
| Ca2 + Overload - | | 7-oxo-3-propyl-1H-pyrazolo[4,3-d]pyrimidin-5-yl) |
| cGMP PDE Antagonists | | phenylsulfonyl]-4-methylpiperazine |
| | Cialis | (6R-trans)-6-(1,3-benzodioxol-5-yl)-2,3,6,7,12,12a- |
| | | hexahydro-2-methyl-pyrazino[1′,2′:1,6]pyrido[3,4-b] |
| | | indole-1,4-dione |
| | Levitra | 4-[2-Ethoxy-5-(4-ethylpiperazin-1-yl)sulfonyl-phenyl]-9- |
| | | methyl-7-propyl-3,5,6,8-tetrazabicyclo[4.3.0]nona-3,7,9- |
| | | trien-2-one |
| | Sulindac and Derivatives | {(1Z)-5-fluoro-2-methyl-1-[4-(methylsulfinyl) |
| | | benzylidene]-1H-indene-3-yl}acetic acid |

TABLE 3
SECONDARY APOPTOTIC TARGETS - Over-the-Counter Supplements
TABLE 3 - SECONDARY APOPTOTIC TARGETS - Over-the-Counter Supplements

| SECONDARY APOPTOTIC | DRUG/CHEMICAL | DRUG/CHEMICAL |
|---|---|---|
| TARGET | COMMON NAME | CHEMICAL NAME |
| Endoplasmic Reticulum | IP6, IP5 | Inositol-1,2,3,4,5,6-hexakisphosphate (Inositol |
| Ca⁺⁺ Overload - | | Hexaphosphate), myo-Inositol 1,3,4,5,6- |
| IP₃-Receptor Antagonists | | pentakisphosphate, (Inositol Pentaphosphate) |
| Endoplasmic Reticulum | Ca²⁺ | Calcium Citrate |
| Ca⁺⁺ Overload - | Vitamin D3 | Cholecalciferol |
| Plasma Membrane Ca⁺⁺ Channel | | |
| Agonists | | |
| Warburg Metabolic Antagonists | DCA | Sodium di-chloro-acetate |

In as much as DCA reverses the Warburg effect and thus changes the sensitivity threshold for Ca²⁺-dependent release of mitochondrial cytochrome C into the cytoplasm and consequent activation of caspase apoptotic mediators, this compound is claimed to be usable to potentiate the actions of either IP6 or Ca²⁺ plus Vitamin D3 either alone or in various combinations. This allows the use of DCA clinically at sub-toxic levels as well as shortening treatment duration for effective induction of apoptosis in malignant cells.

### EXAMPLES

The following examples are provided for illustrative purposes only and are not intended to limit the scope of the invention.

### Example 1

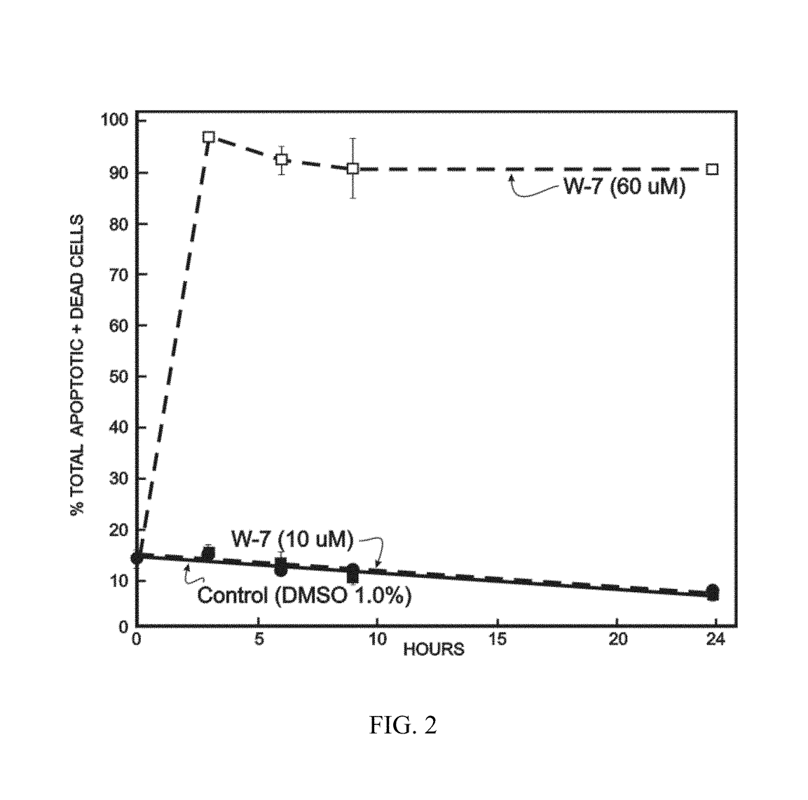

FIG. 2. Shows the Time Course of Effect of the Calmodulin Antagonist W-7 on Induction of Apoptosis in MEL-STR Cells. Malignant (MEL-STR) and non-malignant (MEL-STVP) cells used in these experiments were derived originally from normal human foreskin melanocytes obtained from the Weisberg lab. These original surgical samples were genetically modified to grow continuously in vitro and are non-tumor-forming in a nude mouse model. These same cells were genetically modified further to generate the tumor-forming cell line and were propagated. Thus, this represents an ideal pair of highly similar cells by which drug specificity between normal and malignant cells can be assessed.

Transformed MEL-STR cells were incubated over a period of 24 hrs in the presence of a previously determined ineffective concentration (10 μm) of the CAM antagonist W-7 or the drug vehicle DMSO (1%) as controls and a concentration of 60 μm W-7 as illustrated. Apoptotic+dead cells were assayed in this experiment and those that follow below on a Becton-Dickenson flow cytometer using an Annexin V-FITC Apoptosis Detection Kit as described by the manufacturer.

The results in this experiment show the time course for induction of apoptosis in the malignant cell line (measured by the Annexin Assay) by a highly-specific antagonist of the primary intracellular Ca²⁺ receptor, Calmodulin. Calmodulin is known to be required for traverse of late G1, G2, and specific periods during mitosis and coincides with periods of elevated cAMP levels. Surprisingly, induction of apoptosis can be seen as soon as 3 hours of drug exposure. Morphological rounding of cells can be observed microscopically or by changes in FACS light scatter as early as 1 hr. This is to be compared with typical studies on drug-induced apoptosis which require 48-72 hrs. of exposure. This is especially important because patient exposure and unwanted side-effects can be minimized in vivo. Essentially all of the population (at least in excess of 90%) scores positively for apoptosis. Given the ubiquitous function of Calmodulin in every cell of the body, use of the drug (or more potent congeners) has not been previously used for development by the pharmaceutical industry as far too toxic for clinical use. W-7 does induce apoptosis in transformed cells and does so within extremely short term exposure times.

### Example 2

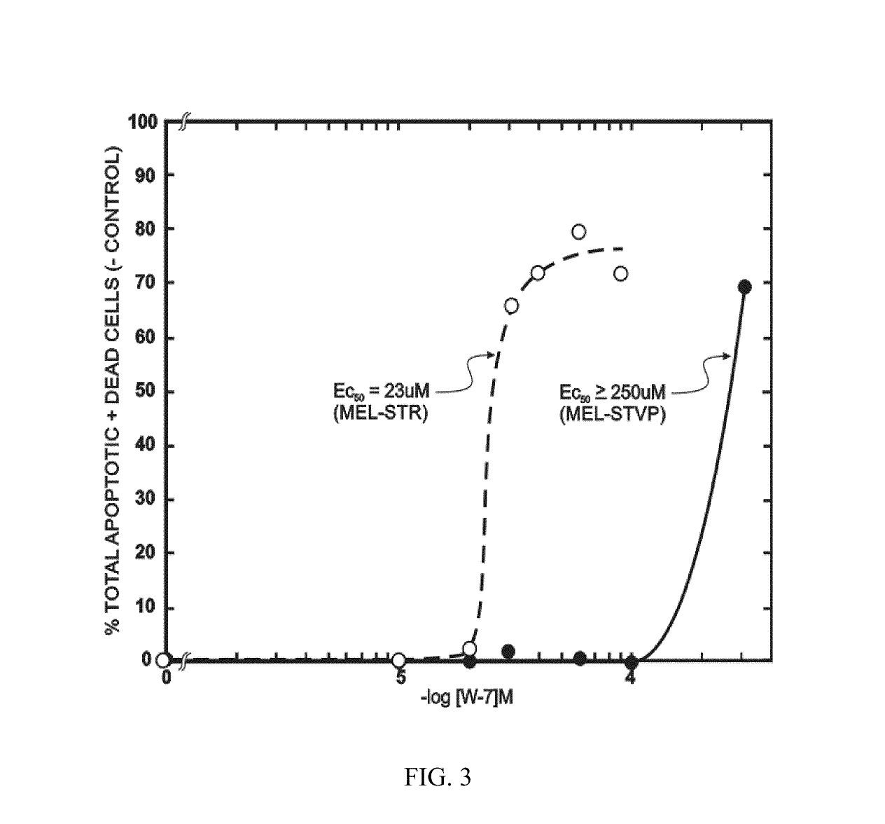

FIG. 3 show a Dose Response Comparison of the Effect of W-7 on Induction of Apoptosis in Malignant MEL-STR and Non-Malignant MEL-STVP Cells. These results are critically important and highly unusual. Transformed malignant cells are more sensitive than non-transformed cells to a completely specific drug which acts only to antagonize a single target, that is, the main IC calcium receptor Calmodulin. The degree of this difference in sensitivity is approximately one order of magnitude. This is large enough that allows the use of W-7 clinically. However, Calmodulin regulates many processes throughout the body and despite this sensitivity advantage may still trigger unwanted side effects. This experiment compares the dose response curves for induction of apoptosis by W-7 in malignant (MEL-STR) and non-malignant (MEL-STVP) human melanocyte-derived cultured cells. The results show that malignant cells are more than 10 times more sensitive to induction of apoptosis than non-malignant cells over a very short 5 hr. exposure time.

### Example 3

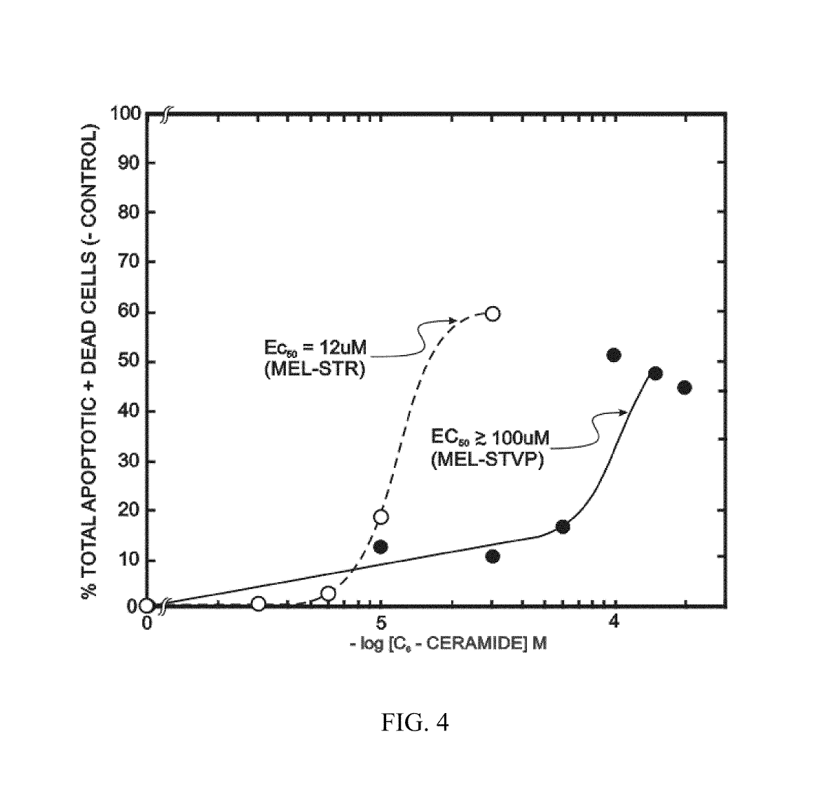

FIG. 4 shows a Dose Response Comparison of the Effect of the PP2A and PKC Agonist C₆C on Induction of Apoptosis in Malignant MEL-STR and Non-Malignant MEL-STVP Cells. The sensitivity difference observed in FIG. 3 is not unique to the Calmodulin antagonist W-7 and a similar order of magnitude in EC₅₀'s is observed using a drug originally thought to stimulate only PKC at the time these experiments were carried out. This drug is now thought to also act to stimulate PP2A as well. These results show that malignant cells exhibit a very significant difference from normal cells in triggering Apoptosis and teach that cancer cells survive by reaching a stable condition of Ca²⁺ overload higher than non-malignant cells. This experiment compares the dose response curves for induction of apoptosis by a putative combined protein kinase C and protein phosphatase 2A agonist, C-6-Ceramide (C6C) in MEL-STR and MEL-STVP cells using a short exposure time of 5 hrs. The results show an approximate 10-fold greater sensitivity of malignant to non-malignant cells to this research drug.

### Example 4

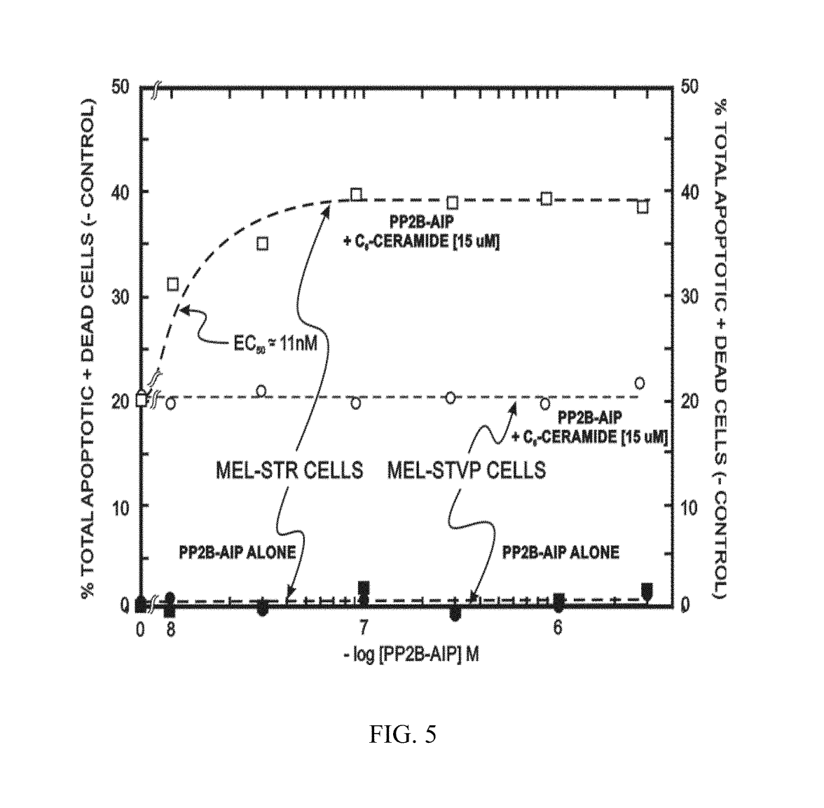

FIG. 5 shows Selective Potentiation of Apoptosis between the Cell Permeable Auto-Inhibitory Peptide PP2B-AIP Antagonist and C₆C in Malignant MEL-STR but not Non-Malignant MEL-STVP Cells. These results illustrate potentiation between C₆C and a completely specific activator of the Calmodulin plus Ca²⁺-requiring enzyme PP2B which is a cell-permeable auto-inhibitory polypeptide (abbr. PP2B-AIP) which acts to block the catalytic site of PP2B. Four observations can be drawn from these results. The first and foremost is that this result was predicted as a consequence of the Calcium Storage/Release Hypothesis of Cell Cycle Regulation in 2002. The synergistic interaction with C₆C provides yet another example of convergence of two highly dissimilar targets which share Ca²⁺ in common. This potentiation is seen only in the transformed MEL-STR cell line, not in untransformed MEL-STVP cells, thus providing a third example of differential sensitivity between malignant and non-malignant cells. Lastly, over the concentration range tested, PP2B-AIP exerted no visible induction of apoptosis in either cell line. This experiment tests whether a completely specific and direct inhibitor/antagonist of the Calmodulin-dependent protein phosphatase 2B, namely the Auto Inhibitory Peptide (PP2B-AIP), can interact synergistically with C6C. This experiment tests whether there is any difference in sensitivity between MEL-STR and MEL-STVP to these drug combinations. The results show that there is synergy but only with malignant cells and not non-malignant cells. Because of this synergy and because of the recognized complete specificity of PP2B-AIP against the catalytic subunit of PP2B, both agents are acting by different mechanisms upon a common pool of cellular Ca++ to induce apoptosis differentially in malignant but not non-malignant cells. In addition, this experiment proves that PP2B is one, but not necessarily the only, calmodulin-dependent regulatory enzyme involved in controlling intracellular Ca++ distribution and apoptosis.

In this and other experiments using this protocol, it has never been possible to kill more than 50% of the MEL-STR cells over a 5 hr. exposure. This is in marked contrast to the potent effect of W-7 (FIG. 2). This implicates at least one other target involved in the actions of W-7. This target is likely to be Calmodulin-dependent Protein Kinase II. This enzyme completes the 4^{th} element of the regulatory enzymatic tetrad as illustrated in FIG. 1. Thus, pharmacological manipulation any pair of tetrad enzymes is expected to interact synergistically and be usable in clinical practice (for example, antagonists of Calmodulin effectors, PP2B and CAM-PK II; agonists of PKC and PP2A).

### Example 5

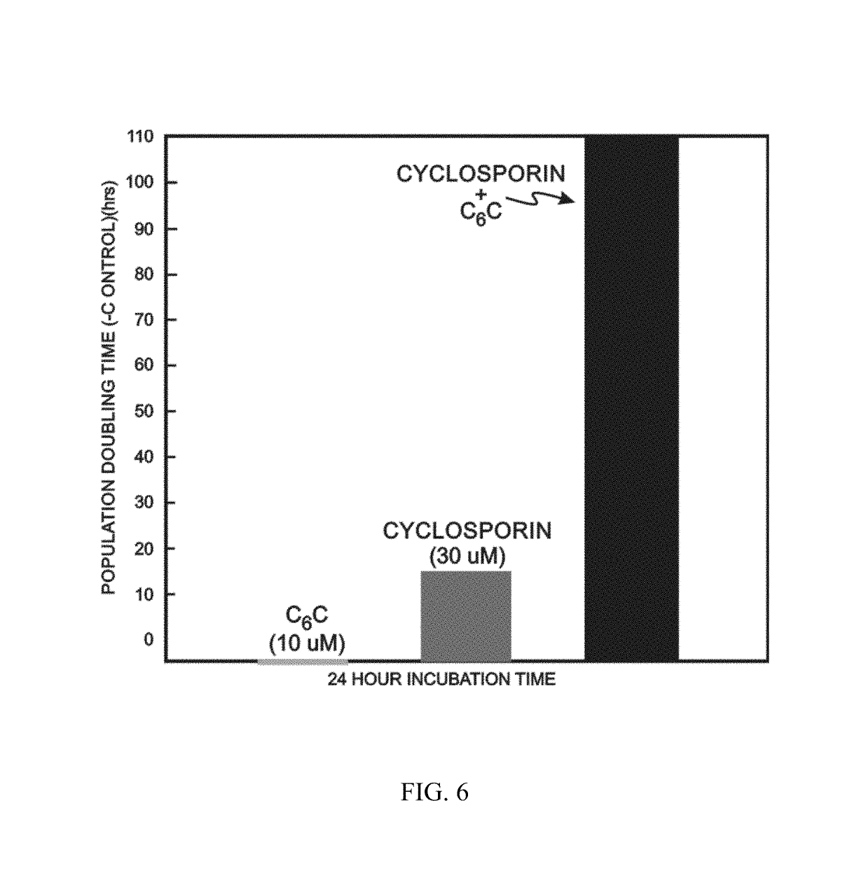

FIG. 6 shows Potentiation of Apoptosis by the PP2B Antagonist Cyclosporin by C₆C in Malignant MEL-STR Cells as Measured by Inhibition of Population Doubling Time. As a test of the specificity of PP2B-AIP, the effect of Cyclosporin (a known inhibitor of PP2B) was tested for pro-apoptotic potential. At quite high concentrations, this compound displayed only slight stimulation of apoptosis or growth inhibition measured in this experiment by a change in doubling time (an indirect assay of cell death). This effect was dramatically potentiated by C6C in MEL-STP cells in the same manner as the previous experiment with PP2B-AIP (FIG. 5). In MEL-STVP cells, no inhibition of cell growth was observed with Cyclosporin at this or lower concentrations nor was there any potentiation observed between these two compounds, thus providing a fourth example of differential sensitivity between malignant and non-malignant cells. This experiment is designed to test the conclusions drawn from the previous experiment in FIG. 5. Here, a structurally different known antagonist of Calmodulin-dependent PP2B (Cyclosporin) is used to test whether potentiation with C6C occurs in MEL-STR cells. Antagonism of PP2B activity can be used to induce apoptosis in malignant cells. In parallel experiments, there was no induction of apoptosis in non-malignant MEL-STVP cells and no potentiation with C6C.

### Example 6

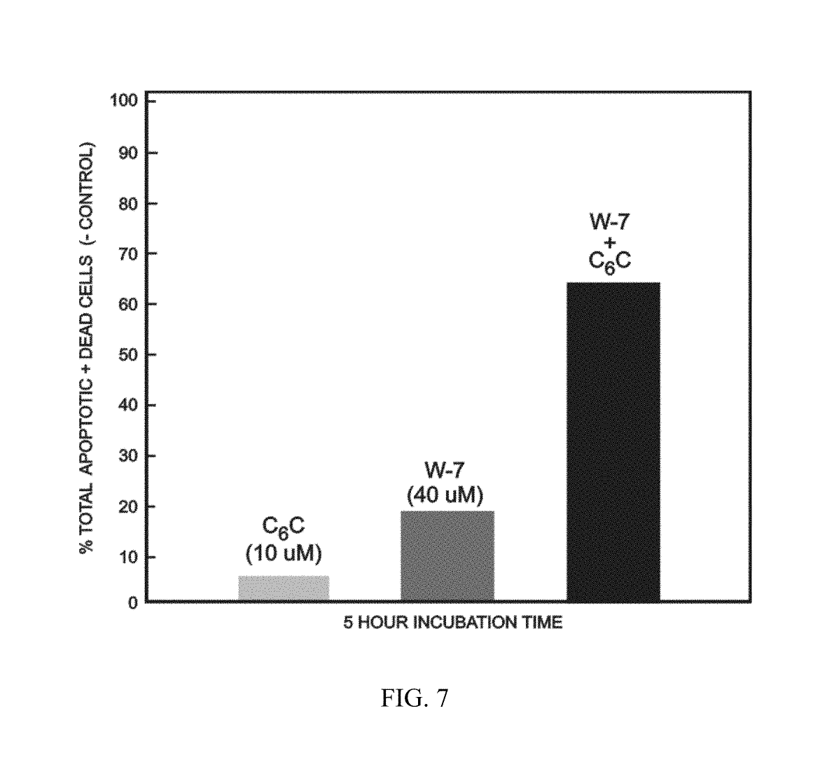

FIG. 7 shows Potentiation Between C₆C and W-7 on Induction of Apoptosis in Malignant MEL-STR Cells. In this experiment, it is shown that when combined with the Calmodulin antagonist (W-7) the effects of this drug on induction of Apoptosis are potentiated. Given the accepted specificity of W-7, this experiment provides evidence that both drugs are affecting processes that share Ca²⁺ in common. The results of the previous two experiments were verified in yet another method. Here, the specific Calmodulin-antagonist, W-7, was tested for apoptotic potentiation in MEL-STR cells in combination with C6C. These two agents are used to synergistically interact to produce an apoptotic response and to do so in a remarkably short 5 hr exposure time.

### Example 7

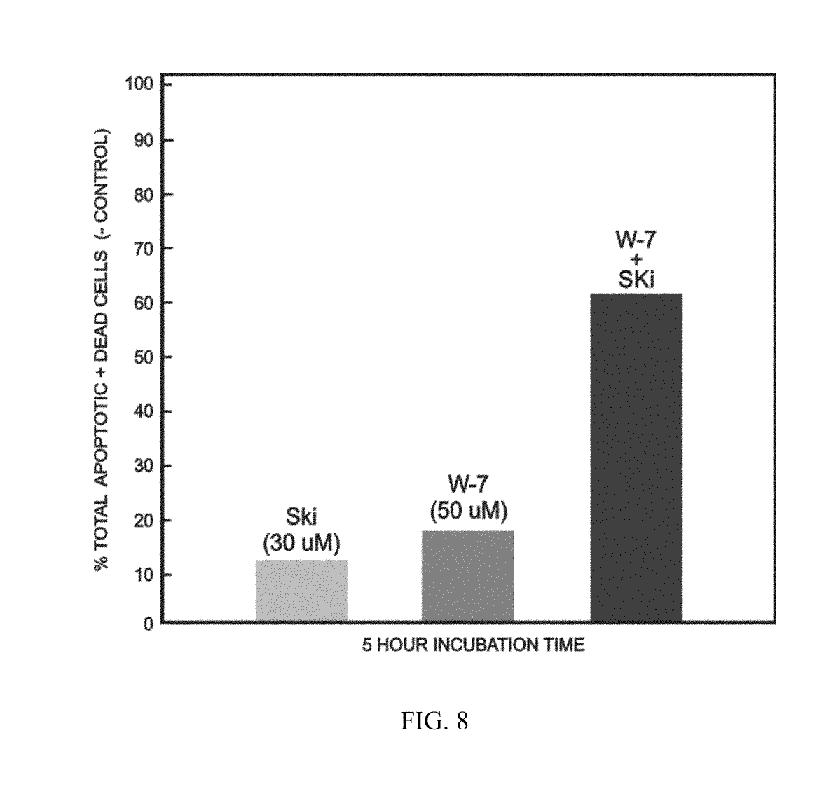

FIG. 8 shows Potentiation between a Sphingosine Kinase Antagonist, SKi (4-[[4-(4-Chlorophenyl)-1,3-thiazol-2-yl]amino]phenol), and W-7 on Induction of Apoptosis in Malignant MEL-STR Cells. This result is especially interesting because it provides evidence that Ca²⁺ must be involved in the action of Sphingosine Kinase. Given the widespread distribution of this enzyme in normal cells, and given the 10-fold potency advantage over non-malignant MEL-STVP cells enjoyed by W-7 (see FIG. 1), these results represent another method for toxicity reduction during cancer therapy and a way of using a drug like W-7 that normally would be expected to be too toxic for use clinically. The synergy between W-7 and SKi provides evidence that both are converging upon a common mediator, namely Ca²⁺. Inhibition of Sphingosine Kinase synergizes with the Calmodulin antagonist W-7 to induce apoptosis in malignant cells.

### Example 8

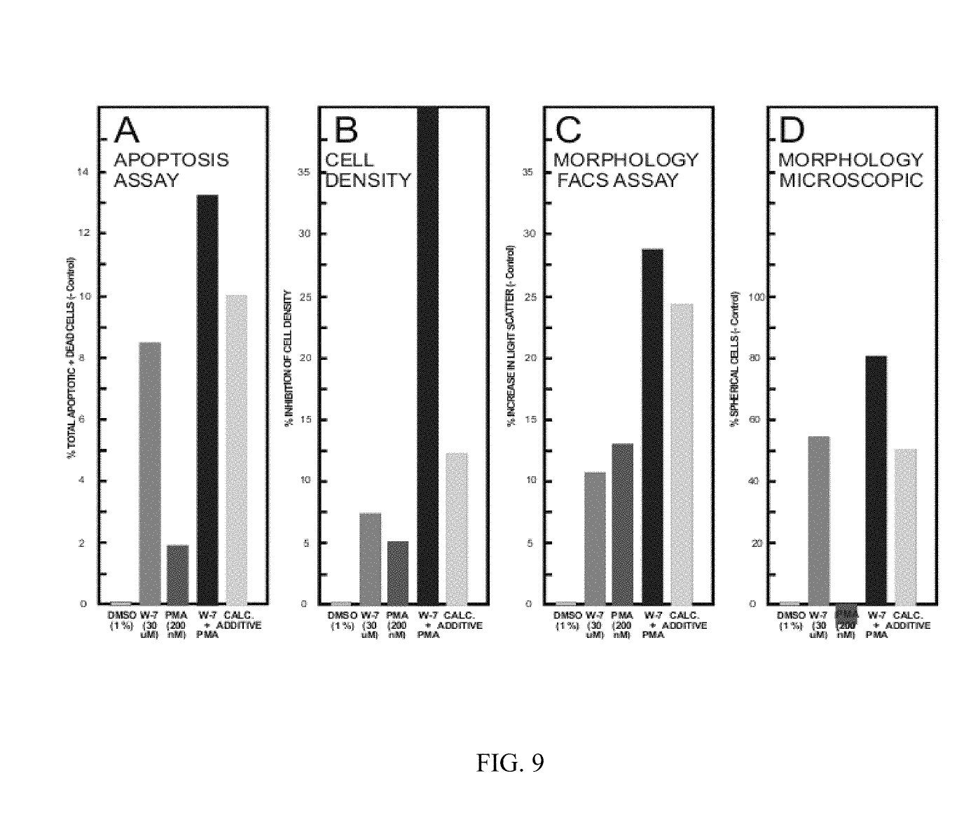

FIG. 9 shows Potentiation between the PKC Agonist PMA and W-7 on Induction of Apoptosis, Growth Inhibition, and Microscopic or FACS Morphology in Malignant MEL-STR Cells. The involvement of a form of PKC in the enzyme tetrad that is involved in Ca²⁺-dependent, cell cycle phase transitions is given in FIG. 4. Here the classic PKC activator, Phorbol Myristate Acetate, is found to be potentiated by W-7. Four different means for assaying the effect of these two chemicals are shown. The standard Annexin assay for detecting Apoptosis shows potentiation (Panel A). However, the more sensitive cell density assay as measured by Coulter Counter shows an even greater potentiation (Panel B). Potentiation is also confirmed by two different measures of cell shape, Light scatter by FACS (Panel C) and by direct microscopic examination (Panel D; results shown are the average of 3 microscope fields selected at random). Throughout all of the results reported here, the morphological effect can be observed as early as 1 hr. drug exposure and has been a sensitive indicator of the subsequent apoptotic fate of the MEL-STR cells under study. It is significant that such morphological shape changes are not observed in MEL-STVP cells. It is also important to realize that the only element in common with PMA and W-7 is Ca²⁺ and these results provide additional evidence supporting the enzymatic tetrad regulatory system in triggering apoptosis.

### Example 9

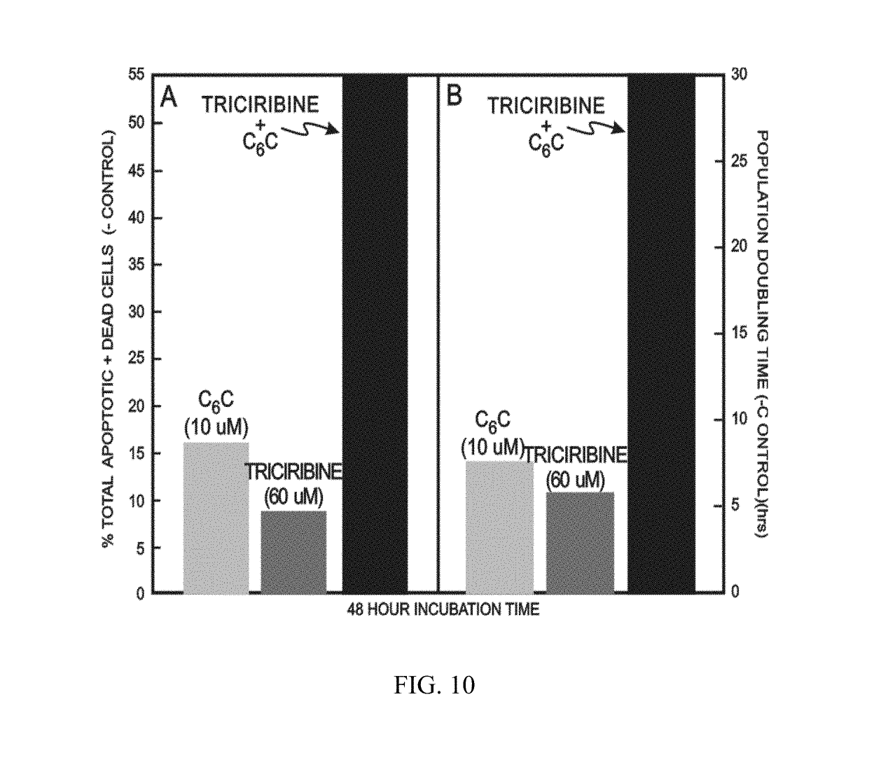

FIG. 10 shows Potentiation of Apoptosis and Inhibition of Growth Rate using an Akt/Protein Kinase B Antagonist (Triciribine) in Combination with C₆C in Malignant MEL-STR Cells. Because PKB has pro-survival/anti-apoptotic properties and, when activated, can overcome checkpoint arrests in both G1 and G2 (periods in which cAMP is normally elevated during traverse of these phases), because cAMP has been implicated in many cells types as an anti-apoptotic agent, and because cAMP is known to stimulate the release of ER Ca²⁺ the question of whether these observations shared a common mechanism involving ER Ca²⁺ reduction was tested experimentally. The effect of low dose C6C on cells treated over a wide concentration range of the PKB inhibitor Triciribine was examined. The results of the highest dose of Triciribine tested are shown in FIG. 8. Clear potentiation was observed consistent with the hypothesis that ER Ca²⁺ overload can promote Apoptosis in cancer cells and that PKB antagonists could be used synergistically with other drugs which modulate cellular Ca²⁺ distribution and as a means of reducing off-target side-effects.

There are other ways of effecting clinical treatment of any and all cancer cell types. For example, any treatment which delivers excess Ca²⁺ to the right location within cells, even on a short term basis, could be combined with an agent that inhibits release of Ca²⁺ from the ER, the obligatory organelle that transfers Ca²⁺ to the mitochondria and induces an apoptotic response. Calcitriol (the active form of Vitamin D) reduces the incidence of certain cancers to a small but significant degree (ca. 17-20%). This cannot be demonstrated when only 400 IU of Vitamin D is taken as a supplement, nor can it be shown when only 1000 mg of Calcium is taken. Only when the two are combined is any effect observed, albeit quite modest. If this regimen is combined with an inhibitor of ER Ca²⁺ release, such as IP6 at doses up to 1000-1600 mg/day, or in another embodiment, at 500-800 mg; taken twice daily, then together this 3-component combination synergistically interacts to produce a much larger reduction of cancer incidence as well as reducing or even eliminating established cancers. Below are two prophetic examples illustrating different forms of cancer and the responses that can be expected as measured by antigen markers.

### Example 10

Since this 3-part regimen, at the levels shown, should have no detectable side effects, it may be used in conjunction with either male or female hormone replacement therapies in order to nullify any chance of elevated cancer risk associated with testosterone or estrogen supplementation.

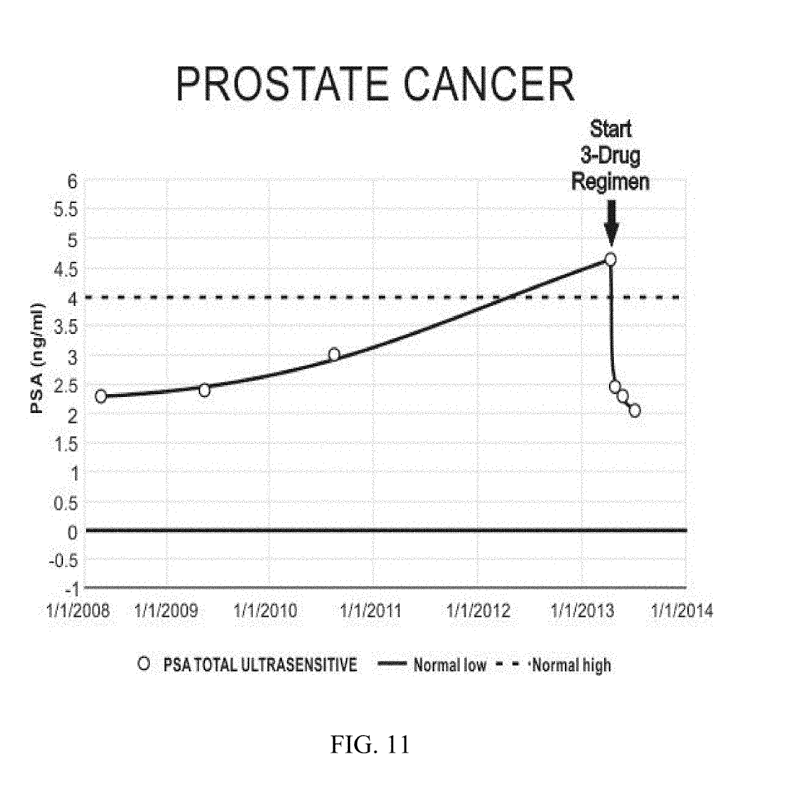

FIG. 11 shows a Prophetic Example in a Patient Diagnosed with Prostate Cancer and Subjected to a Treatment Regimen Designed to Produce Endoplasmic Reticulum Calcium Overload Using an Over-The-Counter 3 Component Mixture of Agents. A patient is prescribed 1000 mg Calcium Citrate (or mixed salts of citrate, malate, and carbonate), 2000 IU of Vitamin D3, and 500 mg IP6 to be taken twice daily 12 hrs. apart. This regimen is continued for at least 6 months. The dose of Calcium salt and IP6 (but not Vitamin D3) can be increased to thrice daily without side effects. This treatment should be combined with adequate exposure to sunlight. Relief of symptoms can be expected within the first 2-3 weeks of treatment and the effects of this regimen can be followed objectively by standard PSA measurements as illustrated in this figure or more specific prostate cancer-specific antigens in development.

### Example 11

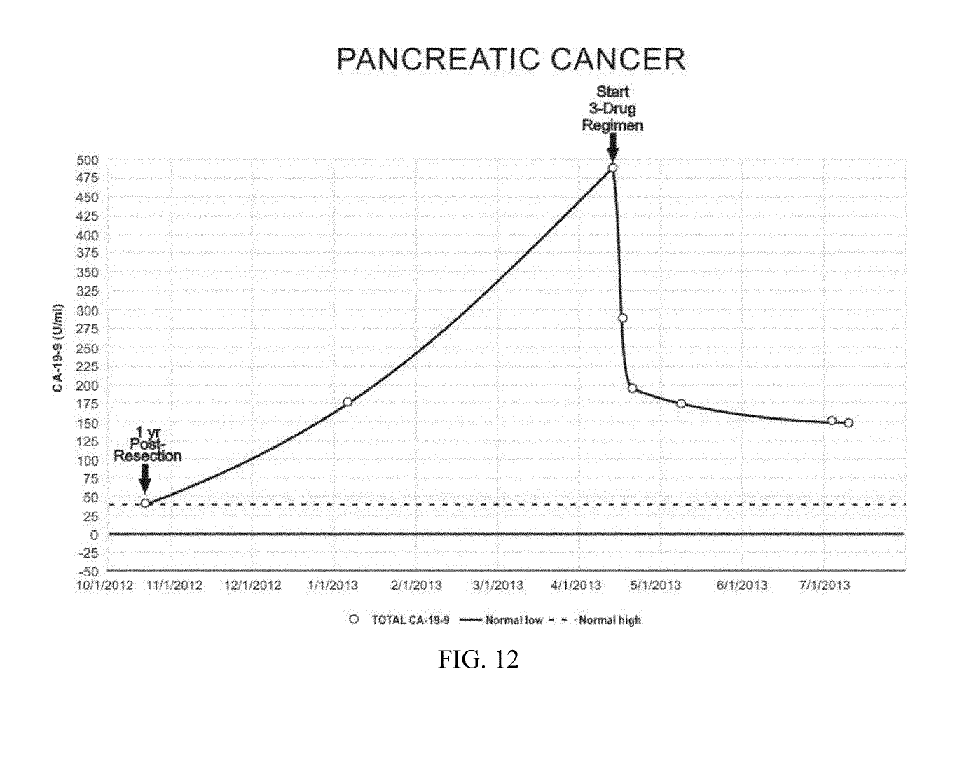

FIG. 12 shows a Prophetic Example in a Patient Diagnosed with Inoperable Metastasized Pancreatic Cancer with a Three Month Survival Estimate and Subjected to a Treatment Regimen Designed to Produce Endoplasmic Reticulum Calcium Overload Using an Over-The-Counter 3 Component Mixture of Agents. The same regimen as described in FIG. 11 is provided. Enlargement of metastasized tumors should be arrested and some tumors may be eliminated entirely. The effect of the treatment regime is illustrated in this figure by radioimmunoassay of the pancreatic cancer antigen CA-19-9 over time.

### Example 12

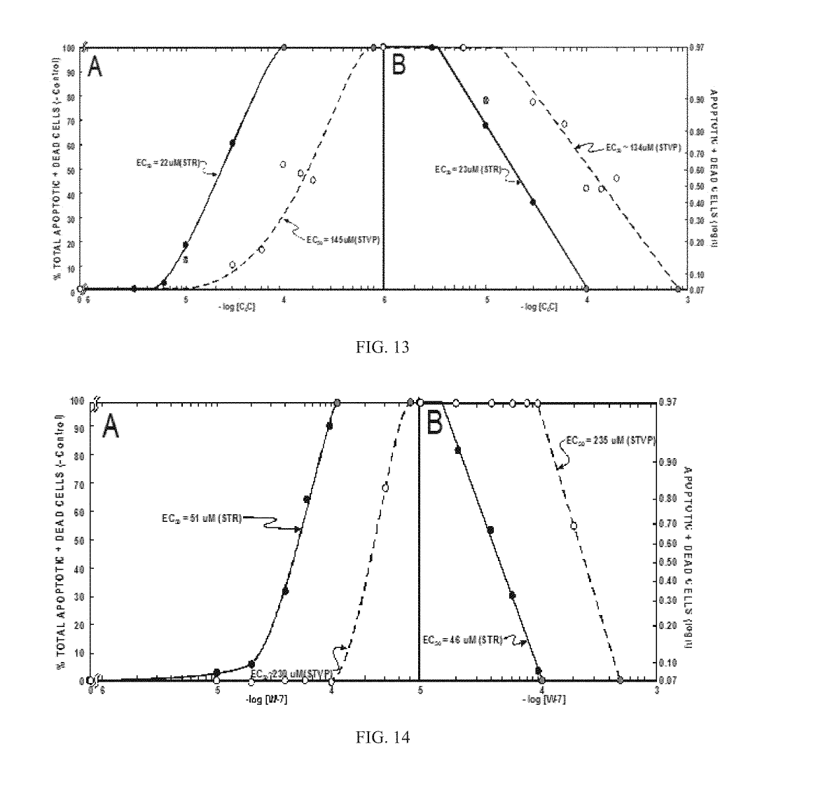

FIG. 13 shows Apoptotic+dead cells were determined by FACS analysis using a commercial Becton-Dickinson Annexin V-FITC/Propidium Iodide apoptosis detection kit. Drug exposure time was 5 hrs. Viable and live cells were measured by FACS and by Coulter counting, respectively. Dose-dependent decreases in viable cells were inversely proportional to the number of Annexin+Propidium Iodide positive staining cells (data not shown) in these and the following experiments. Panels A & B: solid circles and lines, STR cells; open circles and broken lines, STVP cells; Panel B, inverse logit/log plot of the data from Panel A. Solid gray circles in Panel B (extrapolated minimum values on abscissa) were used to plot the maximum values shown in Panel A.

Induction of apoptosis in transformed (malignant) cells is more sensitive to calcium-perturbing drugs than in untransformed cells. This was found to be the case as shown in FIGS. 13 and 14. As shown in FIGS. 13A & B, N-hexanoyl-ceramide (sourced from Sigma or Calbiochem and abbreviated as C₆C—a PKC agonist) is ca. 6-fold more potent at inducing apoptosis in STR compared to STVP cells. Similarly, a Calmodulin antagonist, N-(6-Aminohexyl)-5-chloro-1-naphthalenesulfonamide, HCl (sourced from Calbiochem and abbreviated as W-7), was found to be ca. 5-fold more potent as a pro-apoptotic agent in STR versus STVP cells (FIGS. 14A & B). It is noteworthy that in the case of both of these drugs, induction of apoptosis occurred with only a brief 5 hour exposure period. Based on known effects of PKC isoforms and Calmodulin on cellular calcium pumps, gates, and cytosolic calcium levels, these results are consistent with mechanisms that involve increased ER calcium sequestration.

### Example 13

FIG. 14's Legend is the same as described in FIG. 13.

### Example 14

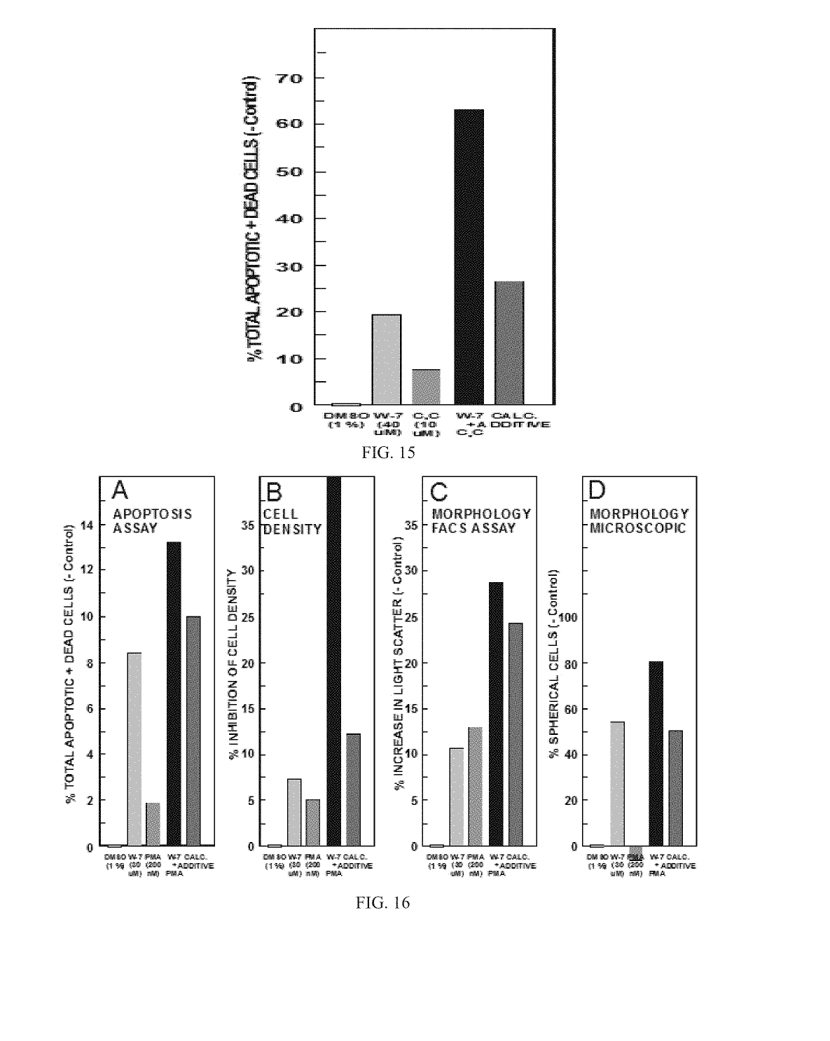

FIG. 15 shows Drug additions and concentrations are as indicated. DMSO was the solvent used for both drugs. Drug exposure time was 5 hours. Rightmost darkest gray bar represent expected values for simple additivity. Control values for apoptotic+dead cells were subtracted from drug-treated values and typically ranged from 8-12% from experiment to experiment. These two drugs interact synergistically as inducers of apoptosis in STR cells (see FIG. 15). These results indicate that both drugs act upon a common final pathway leading to apoptosis

### Example 15

FIG. 16's legend is the same as described in FIG. 16. Panel B, cell density measured by Coulter counting; Panel C, morphological changes measured by changes in forward light scatter as measured by FACS; Panel D, percentage of spherical cells was determined on live cell populations by counting approximately 500 cells in a representative field using an inverted phase contrast microscope. Using a more classical PKC agonist such as phorbol myristate acetate (sourced from Calbiochem and abbreviated as PMA), synergy between this drug and submaximal concentrations of W-7 was also observed (see FIG. 16A). These synergistic drug effects on induction of apoptosis could also be demonstrated by a variety of different assay methods such as elimination of cells (as measured by Coulter counting, see FIG. 16B) and morphology (as measured by forward light scatter using FACS, see FIG. 16C, or, microscopically as cells changing from a flattened to spherical shape, see FIG. 16D).

### Example 16

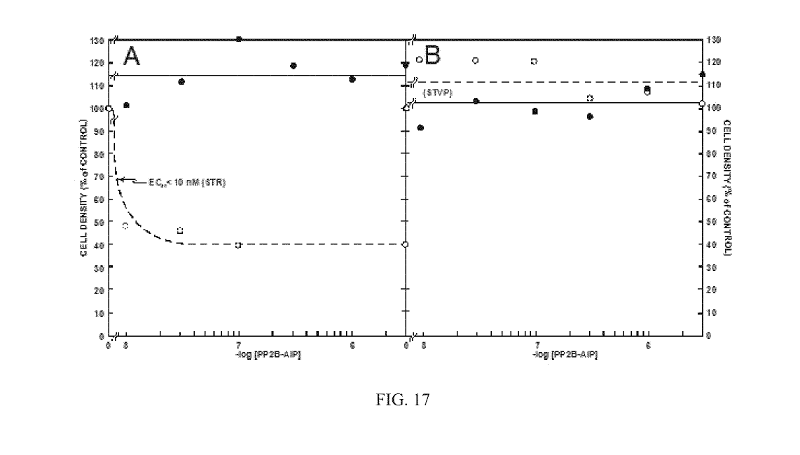

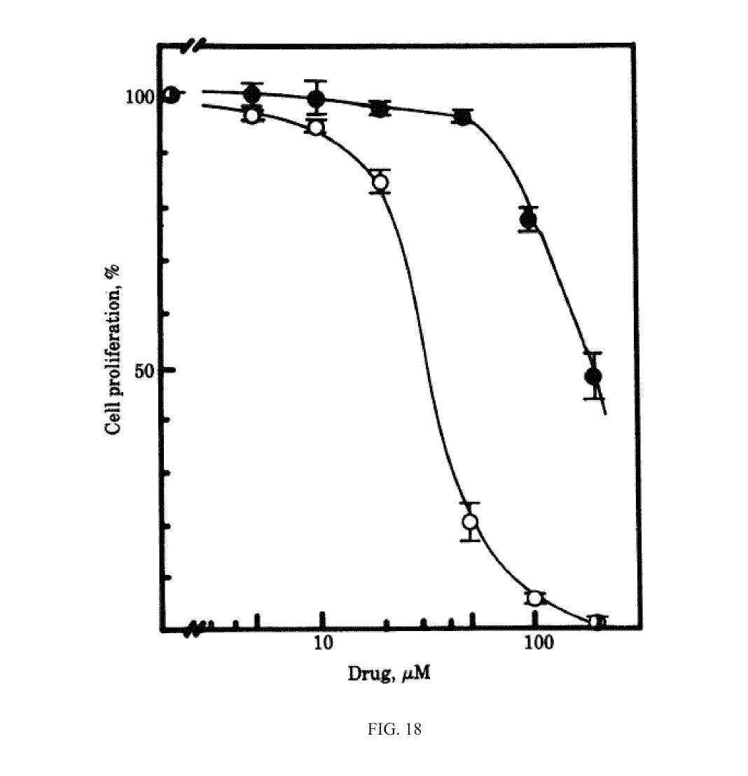

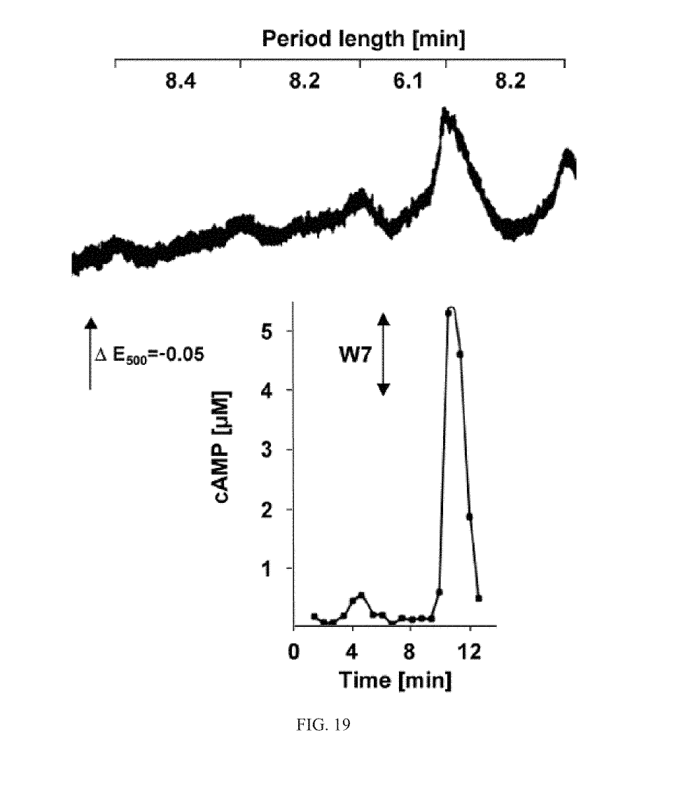

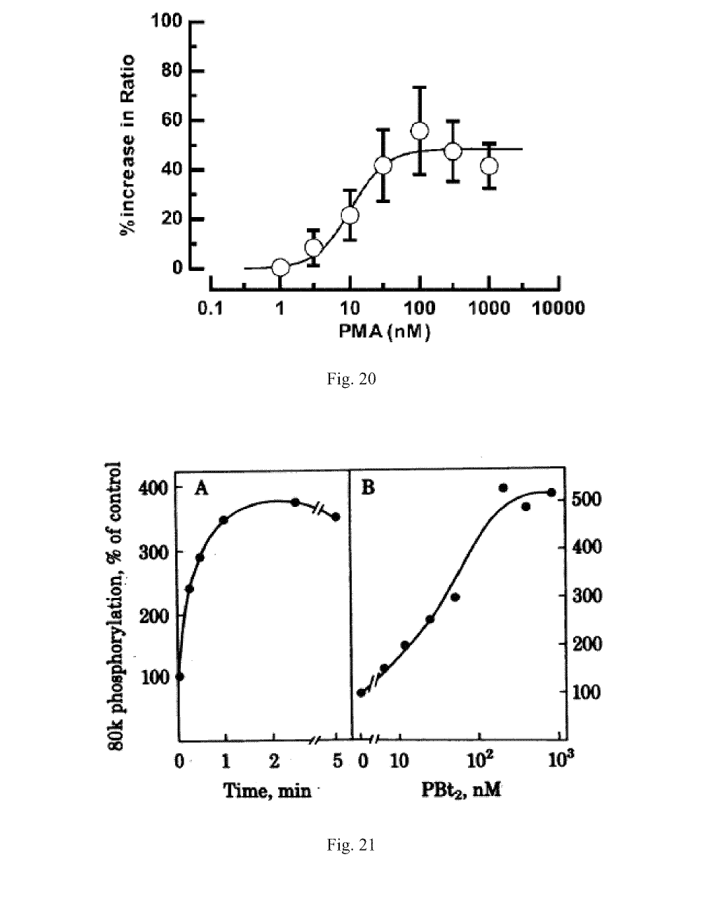

FIG. 17 shows Panel A, STR cells; Panel B, STVP cells. Closed circles, solid lines, PP2B-AIP only; open circles, dashed lines, PP2B+C6C (15 uM). Cell density was quantified by Coulter counting which measures only cells with Panel A, STR cells; Panel B, STVP cells. Closed circles, solid lines, PP2B-AIP only; open circles, dashed lines, PP2B+C6C (15 uM). Cell density was quantified by Coulter counting which measures only cells with ionically intact plasma membranes (presumed live cells). Drug exposure time was 24 hours. Cell density values are expressed as a percentage of maximum for no drug or C6C only controls. Solid black lines in Panels A & B and dashed line in Panel B represent the average of all values across the PP2B-AIP concentration range ionically intact plasma membranes (presumed live cells). Drug exposure time was 24 hours. Cell density values are expressed as a percentage of maximum for no drug or C6C only controls. Solid black lines in Panels A & B and dashed line in Panel B represent the average of all values across the PP2B-AIP concentration range. The ability of PKC agonists on apoptosis induction is potentiated by antagonists of Calmodulin-dependent protein phosphatase (a.k.a. Calcineurin or PP2B) and these effects occur at lower drug concentrations in transformed cells than in untransformed cells. Accordingly, the effects of a completely specific inhibitor of PP2B (cell permeable, PP2B auto-inhibitory peptide, abbreviated as PP2B-AIP and sourced from Calbiochem) were examined with and without submaximal concentrations of C₆C in both STR and STVP cells. The results are illustrated in FIGS. 17A & B). In these experiments, PP2B-AIP alone had no discernible effect on the growth of either STR or STVP cells over a wide concentration range. However, in the presence of submaximal C₆C concentrations, PP2B-AIP was converted into a highly potent (EC₅₀˜10 nM) and effective growth inhibitor (and inducer of apoptosis, results not shown) of STR but not STVP cells.

The manipulation of intracellular calcium distribution, using specific and predictable submaximal, synergistic drug combinations, can be employed to selectively eliminate malignant cells while sparing normal cells. To the extent that the underlying mechanisms for these effects
a) represent obligatory and fundamental regulatory pathways for cell cycle progression,
b) are effective over short exposure times, and
c) are resistant to mutational escape processes, the experimental approach demonstrated here teaches a clinical approach that is applicable to every form of malignancy.

The description of the various embodiments has been presented for purposes of illustration and description, but is not intended to be exhaustive or limiting of the invention to the form disclosed. The scope of the present invention is limited only by the scope of the following claims. Many modifications and variations will be apparent to those of ordinary skill in the art. The embodiments described and shown in the figures were chosen and described in order to explain the principles of the invention, the practical application, and to enable others of ordinary skill in the art to understand the invention for various embodiments with various modifications as are suited to the particular use contemplated. All references cited herein are incorporated in their entirety by reference.

What is claimed is:

1. A method for treating a cancer in a patient comprising administering to said patient effective amounts of two or more drugs at concentrations which interact synergistically, that stimulate an increase in the Ca²⁺ burden of smooth endoplasmic reticulum and mitochondria wherein the drugs are administered at less than each drug's respective EC₅₀ values.

2. The method of claim 1 wherein at least one of said drugs stimulates Smooth-Endoplasmic-Reticulum Ca-ATPase (SERCA) and wherein at least one of said drugs is an antagonist of Smooth-Endoplasmic-Reticulum (SER) Ca²⁺ gates.

3. The method of claim 1 wherein at least one of said drugs is selected from the group consisting of inhibitors of SER IP₃-sensitive Ca²⁺ gates and SERCA agonists, and one of said drugs are selected from the group consisting of drugs which are stimulators of particulate guanylate cyclase (pGC).

4. The method of claim 1 wherein at least one of said drugs is selected from the group consisting of inhibitors of SER IP₃-sensitive Ca²⁺ gates and agonists of SERCA and wherein at least one of said drugs is an effective elevator of cyclic guanosine monosphosphate (cGMP) levels including activators of pGCs and inhibitors of cGMP phosphodiesterases (cGMP-PDEs).

5. The method of claim 1 wherein at least one of said drugs is a calmodulin (CAM) antagonist, including antagonists of the CAM targets calcineurin/protein phosphatase 2B (PP2B) and CAM-dependent protein kinase II (CAM-PKII) and wherein at least one of said drugs is a Protein Kinase C (PKC) agonist.

6. The method of claim 1 wherein at least one of said drugs is a PKC agonist and wherein at least one of said drugs is an inhibitor of cGMP-PDEs.

7. The method of claim 1 wherein at least one of said drugs is a PKC agonist and wherein two additional drugs of the classes CAM-PKII antagonists and PP2B antagonists are combined, wherein the drugs are administered at less than each drug's respective EC₅₀ values.

8. The method of claim 1 wherein at least one of said drugs is a CAM-PKII antagonist and wherein at least one of said drugs is a PP2B antagonist.

9. A method for treating a tumor in a patient comprising administering to said patient effective amounts of two or more drugs that stimulate mitochondrial Ca²⁺ loading.

10. The method of claim 1 wherein the drugs comprise W-7 and C₆C at wherein the drugs are administered at less than each drug's respective EC₅₀ values.

11. The method of claim 1 wherein the drugs comprise W-7 and C₆C; PMA; or SKi.

12. The method of claim 1 wherein the drugs comprise PP2B Antagonist (PP2B-AIP) and C₆C.

13. The method of claim 1 wherein the drugs comprise Cyclosporin A and C₆C.

14. The method of claim 1 wherein the drugs comprise an Akt/Protein Kinase B Antagonist and C₆C.

15. The method of claim 1 wherein the drugs comprise calcium, vitamin D and IP₆.

16. The method of claim 1 wherein one drug is selected from a primary apoptotic target and one drug is selected from a secondary apoptotic target.

17. The method of claim 1 wherein the drugs comprise DCA and W7; or PKC agonist.

18. The method of claim 1 wherein there are at least three drugs.

19. A method for inducing apoptosis in tumor cells comprising administering to said tumor cells two or more drugs wherein the drugs interact synergistically; wherein the drugs stimulate an increase in the Ca²⁺ burden of smooth endoplasmic reticulum and mitochondria; wherein the drugs are selected from at least one protein kinase C agonists and at least one calmodulin antagonist and wherein the drugs are administered at less than each drug's respective EC₅₀ values.
